(19) United States
(12) Patent Application Publication
(10) Pub. No.: US 2026/0337566 A1
(43) Pub. Date: Oct. 29, 2026

(54) COMPOSITIONS OF XCL EXPRESSING CELLS AND METHODS OF USE THEREOF

(71) Applicant: KING'S COLLEGE LONDON, Greater London (GB)

(72) Inventors: Daniel DAVIES, Greater London (GB); Shraddha KAMDAR, Greater London (GB); Adrian HAYDAY, Greater London (GB)

(21) Appl. No.: 19/697,100

(22) Filed: Jun. 3, 2026

Related U.S. Application Data
(63) Continuation of application No. PCT/IB2024/062204, filed on Dec. 4, 2024.
(60) Provisional application No. 63/606,047, filed on Dec. 4, 2023.

(51) Int. Cl. C12N 5/0783; A61K 40/11; A61K 40/31; A61K 40/35; A61K 40/42; A61K 40/50; A61K 45/06; C07K 14/52; C07K 16/00

(52) U.S. Cl. CPC C12N 5/0636; A61K 40/11; A61K 40/31; A61K 40/35; A61K 40/4236; A61K 40/50; A61K 45/06; C07K 14/521; C07K 16/00; C12N 2510/00

(57) ABSTRACT

Provided herein is an engineered immune cell expressing XCL1(X-C motif chemokine ligand 1) or a functional variant thereof, wherein the immune cell is a gamma delta (γδ) T cell. Also provided are methods of modulating immune function by administering said immune cell, and methods of treating cancer and other immune diseases.

### CROSS-REFERENCE TO RELATED APPLICATIONS

This application is a Continuation of International Patent Application No. PCT/IB2024/062204, filed on Dec. 4, 2024, which claims priority to, and the benefit of, U.S. Provisional Application No. 63/606,047, filed on Dec. 4, 2023, the contents of each of which are herein incorporated by reference in their entireties.

### INCORPORATION-BY-REFERENCE OF SEQUENCE LISTING

The present application is being filed along with a Sequence Listing submitted electronically in XML format. The Sequence Listing file “MIL-042US1_SL.xml” is created on May 14, 2026, which is 3,899 bytes in size; the contents of which are incorporated herein for all purposes.

### BACKGROUND

Gamma delta T cells (γδ T cells) are T cells that express a unique T-cell receptor (TCR) composed of one γ-chain and one δ-chain. Gamma delta T cells are of low abundance in the body, are found in the gut mucosa, skin, lungs and uterus, and are involved in the initiation and propagation of immune responses. Gamma delta T cells resident in normal non-hematopoeitic tissue (e.g., skin) likely conduct immunosurveillance.

Immunotherapy with immune cells like γδ T cells which are genetically modified to express chimeric antigen receptors (CARs), is a promising approach to improve outcomes for cancer patients. Expansion and activation of γδ T cells including CAR expressing γδ T cells requires multiple signals including cytokine mediated signals. XCL1 (Lymphotactin α) and XCL2 (Lymphotactin β) can be produced by γδ T cells and the interaction of XCL1/XCL2 with the receptor XCR1 can induce tolerogenic XCR1⁺ dendritic cells (DCs) to tumor tissues and other local tissues, thereby increasing immune responses against cancer and other immune diseases.

### SUMMARY OF THE INVENTION

The present invention provides among other things, immune cells, compositions and methods relating to immune cells (e.g., γδ T cells) comprising elevated expression of XCL1 or functional variant thereof, particularly Vdelta1 T cells (Vδ1+ T cells), as compared to a control immune cell. Also provided are methods of modulating immune function by administering said immune cell, and methods of treating cancer and other immune diseases.

In one aspect, the present invention provides immune cells comprising elevated expression of a X-C motif chemokine ligand (XCL) as compared to a control immune cell. In one aspect, the elevated expression of XCL1 can be induced by overexpression of XCL1 and/or expression of recombinant XCL1 or functional variant thereof. In particular the immune cells are γδ T cells. The γδ T cells comprising elevated expression of XCL1 or functional variant thereof may be blood derived or tissue (e.g., skin) derived γδ T cells (e.g., Vd1 cells). In one aspect, the elevated expression of XCL in the immune cell is from active and over-expression of XCL and by introducing into the cell a XCL or a functional variant thereof. The immune cell (e.g., a γδ Vd1+ cell) may comprise elevated expression of XCL1 (X-C motif chemokine ligand 1, also known as Lymphotactin α) and/or XCL2 (X-C motif chemokine ligand 2, also known as Lymphotactin β).

In some embodiments, the present invention is directed to an engineered immune cell expressing XCL1 (X-C motif chemokine ligand 1) or a functional variant thereof, wherein the immune cell is a gamma delta (γδ) T cell.

In some embodiments, the γδ T cell is a tissue-derived γδ T cell or a blood-derived γδ T cell.

In some embodiments, the γδ T cell is a tissue-derived γδ T cell. As a non-limiting example, the γδ T cell is a skin-derived γδ T cell.

In some embodiments, the γδ T cell is a blood derived γδ T cell. As non-limiting example, the γδ T cell is derived from PBMC (peripheral blood mononuclear cell).

In some embodiments, the γδ T cell is Vdelta1 (Vδ1+) T cell.

In some embodiments, the immune cell is a human skin-derived Vδ1+ T cell.

In some embodiments, the engineered immune cell expresses an increased level of XCL1 or the functional variant thereof, as compared to the expression level of XCL1 in a control immune cell.

In some embodiments, NKG2C and NKp30 expression is elevated.

In some embodiments, IFNγ, TNFα and TGFβ1 expression is elevated.

In some embodiments, the immune cell has an increased cytolytic reaction.

In some embodiments, the engineered immune cell further expresses at least one chimeric antigen receptor (CAR).

In some embodiments, the immune cell has greater cytotoxicity, potency, proliferation and/or anti-tumor activity relative to a control immune cell.

In some embodiments, the XCL1, or functional variant thereof is expressed under an inducible promoter.

In some embodiments, the XCL1 or the functional variant thereof is expressed under a constitutive promoter.

In some embodiments, the γδ T cell is isolated from a tissue or derived from a pluripotent stem cell.

In some embodiments, the γδ T cell is derived from blood or skin.

In some embodiments, the immune cell is allogeneic.

In some embodiments, the immune cell is further expanded.

In some aspects, the present invention is directed to an immune cell comprising elevated expression of XCL1 as compared to a control immune cell, wherein the immune cell is a γδ T cell.

In some embodiments, XCL1 is overexpressed in the cell. As non-limiting examples, overexpression of XCL 1 in the immune cell is induced by a signal, e.g., a signal in a tissue environment. In other embodiments, overexpression of XCL 1 in the immune cell is induced by cell expansion.

In some embodiments, the immune cell is engineered to express a XCL1 or a functional variant thereof.

In some embodiments, the γδ T cell is a blood derived or tissue-derived γδ T cell.

In some embodiments, the γδ T cell is Vdelta1 (Vδ1+) T cell.

In some embodiments, the γδ T cell is expanded.

In some aspects, the present invention is directed to a composition comprising an engineered immune cell described previously, or an immune cells comprising elevated expression of XCL1.

In some aspects, the present invention is directed to a method of modulating immune function comprising administering to a subject in need thereof an engineered immune cell or immune cell comprising elevated expression of XCL1 described previously, or the composition described previously.

In some embodiments, administration of the immune cell leads to increased proliferation of immune cells, cytotoxicity, potency and/or tumor killing.

In some embodiments, the engineered immune cell or immune cell comprising elevated expression of XCL1 activates XCR1+ dendritic cells (cDC1), T cells and/or NK cells. As non-limiting examples, the engineered immune cell or immune cell comprising elevated expression of XCL1 attracts cDC1), T cells and/or NK cells to a tissue.

In some embodiments, the engineered immune cell or immune cell comprising elevated expression of XCL1 increases immunosurveillance in the subject.

In some embodiments, the method comprises administering to a patient in need thereof a therapeutically effective amount of an engineered immune cell or immune cell comprising elevated expression of XCL1 as previously described or the composition as previously described.

In some embodiments, the administration increases recruitment of cDC1 cells to tumor tissues, thereby increasing anti-cancer immune response.

In some embodiments, the patient is further treated with another anti-cancer therapy.

### BRIEF DESCRIPTION OF DRAWINGS

FIG. 1A-1B: Principal Component Analysis (PCA) of samples submitted for bulk RNA sequencing demonstrating that anatomical location impacts T cell transcriptomic signatures. FIG. 1A shows PCA of samples derived from blood and skin. FIG. 1B shows PCA of samples derived from skin and gut.

FIG. 2A-2C: Heat maps depicting expression of genes associated with Tissue-derived memory cells derived from blood and skin Vd1 cells and gut Vg 5, 8, 9, cells. Numbers indicate average expression per group as indicated. N=4/group. FIG. 2A shows differential gene expression in blood versus tissues. FIG. 2B shows genes upregulated in skin versus blood and gut. FIG. 2C shows genes upregulated in the gut versus blood and skin.

FIG. 3A-3C: Skin Vd1 cells exhibit a Tc1-type cytokine profile and the potential to influence downstream adaptive responses via XCL1. N=4/group. FIG. 3A shows box plots depicting expression levels of genes encoding cytokines/chemokines in cells from blood, skin and gut as indicated. FIG. 3B shows a line graph depicting XCL1 expression in paired samples as indicated. * p<0.05, *** p<0.001. FIG. 3C shows a line graph depicting XCL1 expression as measured by ELISA.

FIG. 4A-4G: NK-markers and cytolytic profiles. FIG. 4A-4B shows box plots depicting expression levels of genes encoding NK receptors in cells from blood, skin, and gut (as indicated). N=4/group. FIG. 4C-4D depicts summary data of flow cytometry analysis showing percentages of corresponding positive cells determined after gating Vd1+ cells (left panel) and representative flow plots for one donor (right panel). FIG. 4E shows box plots depicting expression levels of genes encoding Granzyme B and Perforin from blood, skin, and gut as indicated. N=4/group. FIG. 4F depicts summary data of flow cytometry analysis of CD107a positive cells determined after gating Vd1+ cells upon PMA/Ionomycin stimulation. FIG. 4G depicts in the top row representative flow plots upon PMA/Ionomycin stimulation while BFA-only controls are shown in the bottom row.

### DETAILED DESCRIPTION

### Definitions

In order for the present disclosure to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, the term refers to a range of values that fall within 10% of the stated reference value. In some embodiments, the term refers to a range of values that fall within 5% of the stated reference value. The term “between” includes the values of the specified boundaries and all intervening values and fractions.

Allogeneic: As used herein, the term “allogeneic” refers to non-autologous cells, i.e., cells of the same species that differ genetically to the cell in comparison.

Comprise: As used herein, the term “comprises” and “comprising” and variations thereof (e.g., “comprises/comprising,” “includes/including”) should be understood to imply the inclusion of a stated component, feature, element or step or group of components, features, elements or steps but not the exclusion of any other component, feature, element or step or group of components, features, elements or steps. Any one of the terms “comprising,” consisting essentially of, “and” consisting of may be substituted with either of the other two terms, while retaining their ordinary meaning.

Functional variant: As used herein, the term “functional variant” or “functional fragment” refers to a variant that substantially includes the amino acid sequence of a parent, but, compared with the parent amino acid sequence, contains at least one amino acid modification (i.e., substitution, deletion, or insertion), provided that the variant retains the biological activity of the parent amino acid sequence. For example, the amino acid modification is a conservative modification. These conservative modifications include amino acid substitution, addition, and deletion. The modifications can be introduced into a parent protein, e.g., XCL1/2 of the present disclosure, by standard techniques known in the art, such as site-directed mutagenesis and PCR-mediated mutagenesis. The conservative amino acid substitution is a substitution in which the amino acid residue is replaced with an amino acid residue having a similar side chain. Amino acid residue families having a similar side chain have been defined in the art, including basic side chain (e.g., lysine, arginine, histidine), acidic side chain (e.g., aspartic acid, glutamic acid), uncharged polar side chain (e.g., glycine, asparagine, glutamine, serine, threonine, tyrosine, cysteine), nonpolar side chain (e.g., alanine, valine, leucine, isoleucine, proline, phenylalanine, methionine, tryptophan), β-branched side chain (e.g., threonine, valine, isoleucine), and aromatic side chain (e.g., tyrosine, phenylalanine, tryptophan, histidine). The conservative modifications may be selected, for example, based on polarity, charge, solubility, hydrophobicity, hydrophilicity, and/or similarity in amphiphilic properties of residues involved. Thus, the “functional variant” or “functional fragment” has at least 75%, preferably at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the parent amino acid sequence, and retains the biological activity, e.g., binding activity, of the parent amino acid.

Identical: As use herein, the term “identical” in the context of polynucleotide and nucleic acid sequences, refers to a first sequence (e.g., an amino acid sequence or a nucleic acid sequence) that contains a sufficient or minimum number of amino acid residues or nucleotide residues that are i) identical to, or ii) conservative substitutions of aligned amino acid or nucleotide residues in a second amino acid sequence or in a second nucleic acid sequence such that the first and second amino acid sequences or nucleic acid sequences can have a common structural domain and/or common functional activity. For example, amino acid sequences contain a common structural domain having at least about 70%, 75%, 80%, 85%, 90%, 91%, 92%, 93%, 94%, 95%, 96%, 97%, 98% or 99% identity to a reference amino acid sequence, e.g., a sequence provided herein. In another example, nucleic acid sequences have at least about 70%, 75%, 80%, 85%, 90%, 91%, 92%, 93%, 94%, 95%, 96%, 97%, 98% or 99% identity to a reference nucleic acid sequence, e.g., a sequence provided herein.

Isolated: As used herein, the term “isolated” substance, such as an isolated nucleic acid, is a substance that is not in its natural environment, although the isolated substance need not be purified. For example, an isolated nucleic acid is a nucleic acid that is not produced or located in its natural or native environment (e.g., a cell). The isolated material may be isolated, fractionated, or at least partially purified by any suitable technique.

Pharmaceutical composition: As used herein, a “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy. In preferred embodiments, a pharmaceutical composition comprises a pharmaceutically acceptable carrier, diluent or excipient and one or more cells comprising elevated expression XCL1 and/or XCL2.

Subject: As used herein, the terms “subject,” “host,” “individual,” and “patient” are as used interchangeably herein to refer to human and veterinary subjects, for example, humans, animals, non-human primates, dogs, cats, sheep, mice, and rats, etc. In some embodiments, the subject is a human. In some embodiments, the subject is a vertebrate, preferably a mammal. Mammals include, but are not limited to, murines, rats, rabbit, simians, bovines, ovine, porcine, canines, feline, farm animals, sport animals, pets, equine, and primate, particularly human. In another embodiment of the present disclosure, the human is a fetus, an infant, a pre-pubescent subject, an adolescent, a pediatric patient, or an adult. In one aspect, the subject is pre-symptomatic mammal or human. In another aspect, the subject has minimal clinical symptoms of the disease. The subject can be a male or a female, adult, an infant or a pediatric subject.

Therapeutically effective amount: As used herein, the term “therapeutically effective amount” means an amount effective, at dosages, frequency of administration and for duration of time necessary to achieve the desired results such that one or more symptoms or biomarkers is improved after treatment.

Treatment: As used herein, the terms “treatment”, “treat” and “therapy”, and synonyms thereof refer to both therapeutic treatment and prophylactic or preventative measures, wherein the object is to prevent or slow down (lessen) a disease, for example cancer. Those in need of such treatment include those already with a disease as well as those prone to getting the disease or those in whom a disease is to be prevented.

The present invention relates to immune cells comprising elevated expression of XCL1 and/or XCL2 as compared to a control immune cell (e.g., a natural immune cell isolated from a subject), and compositions and methods of uses thereof.

### XCL1 and XCL2

The family of type C chemokines, also known as lymphokines, includes two members XCL1 and XCL2. XCL1 (X-C motif chemokine ligand 1, also known as lymphotactin) is a chemokine of CC chemokine family. XCL1 is an unusual chemokine with only one disulfide bond. Like classical chemokines, XCL1 has a free N-terminus of around 10 amino acids (aa), which is followed by a structured core domain of around 60 aa containing a three-stranded antiparallel beta-sheet and a C-terminal alpha-helix (classical “chemokine fold”) and a C-terminal portion of around 20 aa. The C-terminal extension of XCL1 is conserved across species and contains six hydrophobic and six uncharged polar amino acids. The C-terminus is necessary for chemokine activity. The sequence of human XCL1 comprises 114 amino acids (GonBank: AAH69817.1)

(SEQ ID NO: 1)
MRLLILALLGICSLTAYIVEGVGSEVSDKRTCVSLTTQRLPVSRIKTYTI
TEGSLRAVIFITKRGLKVCADPQATWVRDVVRSMDRKSNTRNNMIQTKPT
GTQQSTNTAVTLTG

XCL1 is secreted by prothymocytes, activated NK cells, activated Th1-polarized CD4⁺ T cells, and activated CD8⁺ T cells, γδ T cells, NKT cells and mast cells. It often is co-secreted with IFN-γ, MIP-1α, MIP-1β, and RANTES and is thus part of the Th1 immune defense.

XCL1 signals through its receptor XCR1 and is a chemoattractant for DC cells, T cells and NK cells in vitro and in vivo. The receptor XCR1 belongs to the G protein coupled receptors superfamily and is exclusively expressed on a subset of dendritic cells (DC), the “cross-presenting” DC in mammals including human. XCR1⁺ DC is also commonly referred to as cDC1 (conventional type 1 dendritic cell (conventional DC1, cDC1). Binding of XCL1 to the XCR1 receptor can fix the conformation of XCR1 in an active state, which facilitates the receptor to interact with and activate heterotrimeric G proteins, leading to activation of downstream signaling pathways. XCL1 engagement of XCR1 triggers a variety of cellular responses including chemotaxis and chemokinesis. The XCR1-XCL1 interaction assists in recruitment of cDC1s to tumor tissues or specific microanatomical locations in lymphoid organs for cross presentation and anti-tumor immune responses by cDC1 (Brewitz et al., 2017; and Iborra et al., 2016).

XCL1 can induce anti-tumor immunity in mice synergy with IL-2. There is also evidence that XCL1 may have a role in graft rejection and T-cell-mediated autoimmunity.

The sequence of human XCL2 comprises 114 amino acids (GenBank: NP_003166.1): MRLLILALLGICSLTAYIVEGVGSEVSHRRTCVSLTTQRLPVSRIKTYTITEGSLRAVIF ITKRGLKVCADPQATWVRDVVRSMDRKSNTRNNMIQTKPTGTQQSTNTAVTLTG (SEQ ID NO: 2). The sequence of XCL2 differs only in the two amino acid residues at positions 7 and 8: Asp and Lys in XCL1 and His and Arg in XCL 2. XCL2 is very similar to XCL1 in expression profile, structure and function, for example, as in XCL1, XCL2 also has two interconvertible protein spatial conformations, a monomeric conformation that binds and activates XCR1 and a dimeric conformation that has a higher affinity for hairpin structures in glycosaminoglycans (GAGs). XCL2 also binds to the XCR1 receptor.

In accordance with the present disclosure, the immune cell comprises elevated expression of XCL1 or a functional variant thereof. In some embodiment, the immune cell comprises elevated expression of XCL2 or a functional variant thereof. In some embodiment, the immune cell comprises elevated expression of XCL1 and XCL2 or functional variant thereof.

### Immune Cells Expressing XCL1/XCL2 or Functional Variant Thereof

In one aspect, the present disclosure provides a novel engineered immune cell, which expresses XCL1, XCL2 or a functional variant thereof.

In some embodiments, an immune cell is engineered to express XCL1, or a functional variant thereof. An exemplary XCL1 variant is XCL1 variant disclosed by Tuinstra et al (Biochemistry, 2007, 46(10):2564-2573).

In some embodiments, the expression and/or activity of XCL1 is constitutive expression.

In other embodiments, the expression and/or activity of the XCL1 is conditional expression. For example, the conditional expression is achieved by operably linking the exogenous genes to an inducible, repressible or tissue-specific promoter.

In some embodiments, an immune cell is engineered to express XCL2 or a functional variant thereof. The expression and/or activity of XCL2 is constitutive expression or conditional expression.

In some embodiments, an immune cell is engineered to express XCL1 or a functional variant thereof, and XCL2 or a functional variant thereof.

In some embodiments, the expression level of the XCL1, XCL2 or the functional variant thereof is controlled at particular time or in a particular tissue. In an embodiment, the promoter is an inducible promoter, i.e., a promoter that initiates transcription only in the presence of specific environmental conditions, developmental conditions, or inducers. Such environmental conditions include, for example, tumor acidic microenvironments, tumor hypoxic microenvironments, etc. Such inducers include, for example, doxycycline, tetracycline, or analogues thereof, wherein the analogues of tetracycline include, for example, chlortetracycline, oxytetracycline, demethylchlortetracycline, methacycline, doxycycline, and minocycline. Inducible promoters include, for example, Lac operon sequence, tetracycline operon sequence, galactose operon sequence, or doxycycline operon sequence. In another embodiment, the promoter is a repressible promoter, i.e., the expression of the XCL1 and/or XCL2 in the cell is inhibited or the XCL1 and/or XCL2 is not expressed in the presence of a repressor specific for the repressible promoter. Repressible promoter includes, for example, Lac repressible elements or tetracycline repressible elements. Inducible/repressible expression systems well known to those skilled in the art can be used in the present disclosure, including, but not limited to, Tet-on system, Tet-off system, Cre/IoxP system, etc.

In some embodiments, the XCL1 and/or XCL2 is operably linked to a transmembrane domain, so as to be anchored on the surface of the engineered immune cell to be expressed.

In some embodiment, the XCL1 is an exogenous XCL1. In other embodiments, the XCL2 is an exogenous XCL2.

In some embodiments, the engineered immune cell expresses an exogenous XCL1 polypeptide having an amino acid sequence at least 75% identical to the amino acid sequence represented by SEQ ID NO: 1. In some embodiments, the engineered immune cell expresses an exogenous XCL1 polypeptide having an amino acid sequence at least 80% identical to the amino acid sequence represented by SEQ ID NO: 1. In some embodiments, the engineered immune cell expresses an exogenous XCL1 polypeptide having an amino acid sequence at least 85% identical to the amino acid sequence represented by SEQ ID NO: 1. In some embodiments, the engineered immune cell expresses an exogenous XCL1 polypeptide having an amino acid sequence at least 90% identical to the amino acid sequence represented by SEQ ID NO: 1. In some embodiments, the engineered immune cell expresses an exogenous XCL1 polypeptide having an amino acid sequence at least 95% identical to the amino acid sequence represented by SEQ ID NO: 1. In some embodiments, the engineered immune cell expresses an exogenous XCL1 polypeptide having an amino acid sequence at least 96% identical to the amino acid sequence represented by SEQ ID NO: 1. In some embodiments, the engineered immune cell expresses an exogenous XCL1 polypeptide having an amino acid sequence at least 97% identical to the amino acid sequence represented by SEQ ID NO: 1. In some embodiments, the engineered immune cell expresses an exogenous XCL1 polypeptide having an amino acid sequence at least 98% identical to the amino acid sequence represented by SEQ ID NO: 1. In some embodiments, the engineered immune cell expresses an exogenous XCL1 polypeptide having an amino acid sequence at least 99% identical to the amino acid sequence represented by SEQ ID NO: 1. In some embodiments, the engineered immune cell expresses an exogenous XCL1 polypeptide having an amino acid sequence at least 100% identical to the amino acid sequence represented by SEQ ID NO: 1.

In some embodiments, the immune cell is engineered to express an exogenous XCL2 or a functional variant thereof.

In some embodiments, the engineered immune cell expresses an exogenous XCL2 polypeptide having an amino acid sequence at least 75% identical to the amino acid sequence represented by SEQ ID NO: 2. In some embodiments, the engineered immune cell expresses an exogenous XCL2 polypeptide having an amino acid sequence at least 80% identical to the amino acid sequence represented by SEQ ID NO: 2. In some embodiments, the engineered immune cell expresses an exogenous XCL2 polypeptide having an amino acid sequence at least 85% identical to the amino acid sequence represented by SEQ ID NO: 2. In some embodiments, the engineered immune cell expresses an exogenous XCL2 polypeptide having an amino acid sequence at least 90% identical to the amino acid sequence represented by SEQ ID NO: 2. In some embodiments, the engineered immune cell expresses an exogenous XCL2 polypeptide having an amino acid sequence at least 95% identical to the amino acid sequence represented by SEQ ID NO: 2. In some embodiments, the engineered immune cell expresses an exogenous XCL2 polypeptide having an amino acid sequence at least 96% identical to the amino acid sequence represented by SEQ ID NO: 2. In some embodiments, the engineered immune cell expresses an exogenous XCL2 polypeptide having an amino acid sequence at least 97% identical to the amino acid sequence represented by SEQ ID NO: 2. In some embodiments, the engineered immune cell expresses an exogenous XCL2 polypeptide having an amino acid sequence at least 98% identical to the amino acid sequence represented by SEQ ID NO: 2. In some embodiments, the engineered immune cell expresses an exogenous XCL2 polypeptide having an amino acid sequence at least 99% identical to the amino acid sequence represented by SEQ ID NO: 2. In some embodiments, the engineered immune cell expresses an exogenous XCL2 polypeptide having an amino acid sequence at least 100% identical to the amino acid sequence represented by SEQ ID NO: 2.

In some aspects, the present disclosure provides an immune cell in which XCL1 and/or XCL2 is overexpressed. The expression levels of XCL1 and/XCL2 may be increased at least 1%, 5%, 10%, 20%, 30%, 40%, 50%, 60%, 80%, 1.5 fold, 2 fold, 3 fold, 5 fold, 10 fold or more as compared to a control immune cell (e.g., a natural immune cell isolated from a subject). Without limiting to any theory, the overexpression of XCL1 and/or XCL2 is induced by expansion of the immune cell in vitro or ex vivo.

In some embodiments, the immune cells comprising elevated expression of XCL1 are generated by transfecting the immune cells with a nucleic acid encoding XCL1 or a functional variant thereof. In other embodiments, the immune cells comprising elevated expression of XCL2 are generated by transfecting the immune cells with a nucleic acid encoding XCL2 or a functional variant thereof. The nucleic acid may be a DNA, a RNA. In some embodiments, the nucleic acid is packaged in a non-viral vector (e.g., a plasmid). or viral vector.

In some embodiments, the overexpression of XCL1 and/or XCL2 is achieved through in vitro stimulation during expansion of the cells.

In some embodiments, the immune cells express elevated protein levels relative to a control immune cell. In some embodiments, the immune cells expression of NKG2C is elevated. In some embodiments, the immune cells expression of NKp30 is elevated. In some embodiments, the immune cells expression of NKG2C and NKp30 is elevated. In some embodiments, the immune cells expression of IFNγ is elevated. In some embodiments, the immune cells expression of TNFα is elevated. In some embodiments, the immune cells expression of TGFβ1 is elevated. In some embodiments, the immune cells expression of IFNγ, TNFα, and TGFβ1 is elevated.

In another aspect, the present invention provides an immune cell expressing a CXC chemokine a CC chemokine, and/or a C chemokine.

In some embodiments, the immune cell comprising elevated expression of XCL1 and/or XCL2 is a T cell or a NK cell. In some embodiments, the immune cell is a γδ T cell, e.g., a tissue derived γδ T cell.

In some embodiments, the immune cell is further engineered to express a cell surface molecule that specifically recognizes a ligand that is a chimeric antigen receptor (CAR) or a T cell receptor, preferably, a chimeric antigen receptor.

The chimeric antigen receptor contains a ligand binding domain, a transmembrane domain, a co-stimulatory domain, and an intracellular signaling domain. In some embodiments, the ligand binding domain may be selected from the group consisting of scFv, Fab, single domain antibody, nanobody, antigen binding ligand, recombinant fibronectin domain, anticalin, and DARPIN. Preferably, the ligand binding domain is selected from the group consisting of scFv, Fab, single domain antibody, and nanobody. Detailed descriptions of chimeric antigen receptors are included below.

### Gamma Delta T Cells

γδ T cells have both adaptive and innate characteristics. These cells have the potential to develop immunological memory. At the same time, they rapidly recognize and respond to ubiquitous changes, but release less cytokines for proliferation. The persistence of such cells in large numbers in vivo is often limited to only a few days. In certain embodiments, the engineered γδ T-cell is selected from the group consisting of: a γ9δ2T cell, a δ1T cell, a δ3T cell, or a combination thereof.

In some embodiments, a T-cell expressing a cytokine described herein is a γδ T cell.

A γδ T cell expresses a γδ TCR composed of one gamma chain (γ) and one delta (δ) chain. The γδ TCR is required for γδ T cell function and development. Γδ T cells are non-conventional T cells which show several properties of innate immune cells. Γδ T cells present in blood and tissues with a restricted TCR repertoire and circulate as cells with a pre-activated phenotype thus being able to generate rapid immune responses. In contrast to αβ T cells, γδ T cells exert a direct cytotoxicity and do not require stimulation through CD3ζ in order to initiate target cell killing. Another advantage of γδ T cells is that ex vivo expanded γδ T cells are relatively short-lived with little expansion in vivo, which can help control cytokine release syndrome (CRS) and other adverse events resulting from cytokine expression or CAR armoring cell therapy. In addition, γδ T cells are unlikely to cause graft-versus-host disease (GvHD) as they interact with antigen independent of major histocompatibility complex (MHC)-recognition, permitting use in an allogeneic setting.

In some embodiments, the immune cells comprising elevated expression of XCL1 and/or XCL2 are isolated from a tissue or derived from a pluripotent stem cell.

In some embodiments, the immune cells comprising elevated expression of XCL1 and/or XCL2 are γδ T cells. In some embodiments, the immune cells comprising elevated expression of XCL1 and/or XCL2 are tissue derived γδ T cells. In some embodiments, the immune cells comprising elevated expression of XCL1 and/or XCL2 are isolated from the skin.

In some embodiments, the immune cells comprising elevated expression of XCL1 and/or XCL2 are allogenic.

In some embodiments, the cells are human blood-derived γδ T cells. In some embodiments, the Vδ1+ T cells are derived from a blood sample. The sample includes peripheral blood, umbilical cord blood or fractions thereof, including buffy coat cells, leukapheresis products, peripheral blood mononuclear cells (PBMCs) and low-density mononuclear cells (LDMCs). In some embodiments, the blood sample is peripheral blood or a fraction thereof. In some embodiments, the sample is human blood or a fraction thereof. In some embodiments, the sample is mouse blood or a fraction thereof. In some embodiments, the sample is rat blood or a fraction thereof.

In some embodiments, the cells are human skin-derived γδ T cells.

In some embodiments, the cells are human blood-derived γδ T cells.

In some embodiments, the cells are mouse blood-derived γδ T cells.

In some embodiments, the cells are rat blood-derived γδ T cells.

In some embodiments, the cells are gut-derived γδ T cells.

In some embodiments, the cells are Vdelta1 T cells.

The blood-derived γδ T cells primarily express the delta variable 1 (V81) chain. References to “Vδ1 T cells” refer to γδ T cells with a Vol chain, i.e., Vδ1⁺ T cells. As used herein, the term “delta variable 1” may also be referred to as Vδ1 or Vd1.

In some embodiments, the γδ T cells are Vδ1+ (Vd1+) T cells, e.g., human Vδ1⁺ T cells (also referred to as Vd1+ T cells).

In some embodiments, the present invention provides a Vδ1⁺ T cell comprising a CAR described herein. In some embodiments, the Vδ1+ T cell may comprise one or more cytokines and/or cytokine receptors. In some embodiments, the Vδ1+ T cell may comprise one or more cytokines and/or cytokine receptors in combination with a CAR.

In some embodiments, the present invention provides a Vδ1⁺ T cell comprising a polynucleotide encoding a CAR described herein. In some embodiments, the Vδ1⁺ T cell may comprise a polynucleotide encoding a one or more cytokines and/or cytokine receptors. In some embodiments, the Vδ1⁺ T cell may comprise a polynucleotide encoding a one or more cytokines and/or cytokine receptors in combination with a CAR.

In some embodiments, the present invention provides a Vδ1⁺ T cell comprising a CAR described herein.

In various embodiments, the present disclosure provides methods for modifying γδ T cells, e.g., Vδ1+ T cells using a cytokine and a cytokine receptor. A cell capable of expressing an attenuated CAR according to the invention may be made by transducing or transfecting the cell with CAR-encoding nucleic acid. The cells can be transduced using the viral vectors as described herein or alternatively using technology described in Riet et al. (2013) Meth. Mol. Biol. 969:187-201 entitled “Nonviral RNA transfection to transiently modify T cell with chimeric antigen receptors for adoptive therapy.”

In some embodiments, the method of modifying the Vδ1+ T cells may also include expansion of the Vδ1+ T cells. In some embodiments, the expansion is achieved through the selective increase in number of Vδ1+ T cells and/or through the promotion of survival of Vδ1+ T cells.

In order to achieve sufficient therapeutic doses of γδ T cell compositions, γδ T cells are often subjected to one or more rounds of stimulation, activation and/or expansion.

In a preferred embodiment, polynucleotides are introduced into a γδ T cell by viral transduction. Viral vector systems suitable for introducing a polynucleotide into a γδ T cell include but are not limited to adeno-associated virus (AAV), retrovirus, herpes simplex virus, adenovirus, vaccinia virus vectors for gene transfer. In one embodiment, polynucleotides are introduced into a T cell by AAV transduction.

In one embodiment, polynucleotides are introduced into a γδ T cell by retroviral transduction. In one embodiment, polynucleotides are introduced into a γδ T cell by lentiviral transduction. In one embodiment, polynucleotides are introduced into a γδ T cell by adenovirus transduction. In one embodiment, polynucleotides are introduced into a γδ T cell by herpes simplex virus transduction. In one embodiment, polynucleotides are introduced into a γδ T cell by vaccinia virus transduction.

In various embodiments, Vδ1+ T cells comprising a nucleic acid sequence encoding one or more cytokines or cytokine receptors are provided. Transduced Vδ1+ T cells may be used as immune effector cells.

In some embodiments, cytokine-modified γδ T cells retain the innate anti-cancer properties. In other embodiments, cytokine-modified γδ T cells acquire additional antigen-specific cytotoxicity and proliferative response. Furthermore, cytokine-modified γδ T cells may retain the function of antigen cross presentation to effector T cells. Cytokine-modified γδ T cells can migrate effectively toward tumor cells as unmodified γδ T cells. Cytokine-modified γδ T cells have high efficacy of tumor killing. In some embodiments, cytokine-modified γδ T cells reduce treatment-related morbidity, including on-target, off-tumor effects.

### Chimeric Antigen Receptors

Classic chimeric antigen receptors (CARs) can graft the specificity of, for example, antibody (Ab) to the effector function of a T-cell. Their usual form is that of a type I transmembrane domain protein with an extracellular antigen recognizing region, a hinge region, a transmembrane domain all connected to a compound intracellular region which transmits T-cell survival and activation signals. The compound intracellular region generally comprises one or more co-stimulatory domains and a primary signaling/activation domain; The most commonly used primary activation domain to design a CAR polypeptide is a CD3ζ signaling domain.

CAR modified T cells have been used successfully in the clinic for the treatment of both hematological malignancies (like B cell or granulocyte malignancies) and solid tumors. CAR-T cell-based cancer immunotherapies still need improvement for several important reasons such as cytotoxicity. The present invention develops CARs lacking intracellular primary activation/signaling domain (e.g., intracellular CD3ζ activation domain) with reduced cytotoxicity. The present application specifically provides γδ T cells expressing said CARs in the presence or absence of cytokines. The CARs expressed in γδ T cells (e.g., donor-derived skin γδ T cells) can be used to treat cancer and other immune diseases. γδ T cells expressing a CAR have functionality comparable to those expressing classic CAR including an intracellular CD3ζ activation signaling domain.

For example, a chimeric antigen receptor (CAR) fusion protein comprises from N-terminus to C-terminus: (i) an antigen binding domain, (ii) a hinge region; (iii) a transmembrane domain, and (iv) one or more costimulatory domains. In accordance, a CAR polypeptide does not comprise an intracellular signaling/activation domain. Typically, a CAR polypeptide described herein does not comprise an intracellular signaling domain derived from CD3ζ.

Optionally the CAR expresses one or more additional polypeptides. For example, the CAR expresses one or more cytokine receptors. Polynucleotides and vectors encoding these constructs are further provided herein as well as the polypeptides encoded by them in vitro or in vivo.

In some embodiments, Vd1 T cells or NK cells comprise a CAR, armored with one or more cytokines. In some embodiments, Vd1 T cells or NK cells can comprise a CAR, co expressing one or more cytokines. In some embodiments, Vd1 T cells or NK cells can comprise a CAR, expressing XCL1 and/or XCL2

### Car Components

a. Antigen Binding Domain

CARs can use antigen-binding domain properties to re-direct immune cell specificity and responsiveness to selected targets. The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

In some embodiments, the antigen binding domain may be derived from an antibody or antigen-binding fragment thereof that binds to the antigen. Examples of antibody fragments include Fab, F(ab′)₂, single chain variable fragment (scFv), tandem scFv, BiTE, single domain (sdAb) antibody, nanobody, diabody, single chain diabody, minibody, camelid VHH, fusion protein, triabody, tetrabody, disulfide stabilized Fv protein (“dsFv”), scFv-Fc, multi-specific antibodies formed from antibody fragment; the antibody, or fragment thereof, specific to a disease-associated antigen.

In some embodiments, the antigen-binding domain may be derived from a polypeptide that binds to a disease associated antigen. In some embodiments, the polypeptide may be a receptor or a portion of a receptor that binds to an antigen. In another embodiment, the antigen-binding domain may be derived from a ligand that binds to an antigen.

The antigen can be a tumor associated antigen (TAA). Exemplary tumor associate antigens may include antigens of 4-IBB, 5 AC, 5T4, A2aR, activin receptor-like kinase 1, AGS-22M6, AKAP4, alpha-fetoprotein, angiopoietin 2, B7-H3, BAFF, BAGE, BCR-ABL, BORIS, CA-125, CA19-9, C242 antigen, carbonic anhydrase 9 (CA-IX), CCR4, CD19, CD20, CD22, CD23 (IgE receptor), CD24, CD28, CD30 (TNFRSF8), CD33, CD37, CD38 (cyclic ADP ribose hydrolase), CD40, CD44 v6, CD51, CD56, CD70, CD71, CD73, CD74, CD79B, CD80, CD137, CD140a, CD152, CD200, CD221, CD274, CEA, ch4D5, CLDN18.2, CS1, CSF1R, CTLA-4, C-X-C chemokine receptor type 4, DLL4, DR5, EBAG9, EGF, EGFR, EGFL7, EpCAM, ERBB2, ERBB3, FAP, fibronectin extra domain-B, folate receptor 1, folate receptor alpha, folate hydrolase, Frizzled receptor, GAGE, GD2 ganglioside, GD3 ganglioside, glioma, glypican 3, GP MB, gplOO, GUCY2C, FIERI, HER2/neu, HER3, HGF, HHGFR, histone complex, HLA-DR, human scatter factor receptor kinase, HPV-16, HSP105, IDH1, IDO1, IGF-I, IGF-1 receptor, ILGF2, IL-6, IL-13, integrin αvβ3, integrin α5β1, KIR, LAG-3, Lewis-Y antigen, LY6K, MAGE-1, MAGE-A3, MAGE-C2, MAGE-D4, MAPG, MART-1, Melan-A, MET, MCP-1, mesothelin, MIF, MSLN (Mesothelin), MS4A1, mucin CanAg, MUC1, MUC4, MUC1 6, NG2, N-glycolylneuraminic acid, Notch receptor PD-1, NY-ESO-1, OCAA, PAP, PDGF-R a, PDCD1, PD1, PD-L1, phosphate-sodium co-transporter, phosphatidylserine, PRAME, PSA, RANKL, RON, ROR1, SDC1, Sialyl-Tn, SLAMF7, SPAG-9, SSX1, STEAP1, ostimula, TAG-72, telomerase, TEM1, tenascin C, TGF-β, TFM-3, TLR, TAM, TFM-3, TRAIL-R2, TRAIL-R1, TWEAK receptor, tumor specific glycosylation of MUC1, tumor-associated calcium signal transducer 2, tumor antigen CTAA16.88, TYRP1 (glycoprotein 75), VEGF-A, VEGFR2, VEGFR-1, vimentin, VISTA, WT1, and X AGE-1b; the antibody, or fragment thereof, is directed to a disease-associated antigen which is chosen from 1-40-p-amyloid, AOC3 (VAP-1), ACVR2B, angiopoietin 3, beta-amyloid, C5, CCL11 (eotaxin-1), CCR5, CD2, CD3, CD4, CD5, CD11, CD18, CD20, CD23 (IgE receptor), CD25 (a chain of IL-2 receptor), CD28, CD41 (integrin alpha-lib), CD52, CD125, CD147 (basigin), CD 154 (CD40L), CEA-related antigen, clumping factor A, endotoxin, GMCSF receptor α-chain, growth differentiation factor 8, hemagglutinin, HNGF, Hsp90, IGHE, IgE Fc region, IL-Iβ, IL-4, IL-5, IL-6, IL-9, IL-12, IL-13, IL-17, IL-17A, IL-20, IL-22, IL-23, IL-6 receptor, integrin α4β7, integrin α7δ7, integrin a4, integrin αI3/4β3, interferon α/β receptor, interferon gamma-induced protein, IFN-γ, IFN-a, ITGB2 (CD 18), LFA-1 (CD 11a), LINGO-1, lipoteichoic acid, LOXL2, myelin-associated glycoprotein, myostatin, neural apoptosis-regulated proteinase 1, NGF, NOGO-A, Oryctolagus cuniculus, OX-40, PCSK9, phosphatidylserine, platelet-derived growth factor receptor beta, RANKL, Rhesus factor, sclerostin, SOST, sphingosine-1-phosphate, TFPI, TGF-β, TGF beta 2, TGF beta 1, TNF-a, VEGF-A, and VWF; or the antibody, or fragment thereof.

In some embodiments, the antigen is a B-cell associated antigen.

In some embodiments, the antigen is a T-cell associated antigen. For example, the antigen can be selected from CD3, CD4, CD8, PECAM1, CD103 (naïve, RTEs); CCR7, CD127, CD62L (Tscm, Tcm); IL-2RA (Tcm), HLA-DR, CCR5, TBX21, GZMA (Tem, Teff), CCR, ITGAL, IFNg, IL-13, IL-17A, IL-2, IL-21, IL-22, IL-25, IL-26, TBX21, TCF7, EPCAM.

In some embodiments, the antigen is an antigen associate with a solid tumor.

b. Hinge Domain

A CAR molecule generally includes the structure of the spacer/hinge domain between the antigen binding domain and the transmembrane domain. The hinge domain is a spacer that provides separation of the antigen binding domain (e.g., scFv) from the cell membrane and an intracellular signaling module that mediates T-cell activation. One of ordinary skill in the art will appreciate that a hinge sequence is a short sequence of amino acids that facilitates flexibility (see, e.g., Woof et al., Nat. Rev. Immunol., 4(2): 89-99 (2004)). The hinge sequence can be any suitable sequence derived or obtained from any suitable molecule such as a human protein. In some embodiments, the length of the hinge sequence may be optimized based on the CAR and targeted antigens.

A CAR contemplated herein comprises a hinge sequence between the antigen-binding domain and the transmembrane domain.

In some embodiments, the hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the spacer domain includes at least a portion of an IgG1, an IgG2, an IgG3, an IgG4, an IgE, an IgM, or an IgA immunoglobulin Fc region that falls within its CH2 and CH3 domains. In some embodiments, the hinge domain may also include at least a portion of a corresponding immunoglobulin hinge region. In some embodiments, the hinge is derived from or includes at least a portion of a modified immunoglobulin Fc region, for example, a modified IgG1 Fc region, a modified IgG2 Fc region, a modified IgG3 Fc region, a modified IgG4 Fc region, a modified IgE Fc region, a modified IgM Fc region, or a modified IgA Fc region. The modified immunoglobulin Fc region may have one or more mutations (e.g., point mutations, insertions, deletions, duplications) resulting in one or more amino acid substitutions, modifications, or deletions that cause impaired binding of the hinge domain to an Fc receptor (FcR).

c. Transmembrane Domain

With respect to the transmembrane domain, the CAR can be designed to comprise a transmembrane domain that is fused to the antigen-binding domain of the CAR, e.g., through a hinge sequence. The transmembrane connects the intracellular signaling domain to the hinge region of a CAR. In some embodiments, the transmembrane domain that naturally is associated with one of the domains in the CAR is used. In some embodiments, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Typically, the transmembrane domain denotes a single transmembrane alpha helix of a transmembrane protein, also known as an integral protein.

A suitable transmembrane domain of particular use in an CAR described herein may be a transmembrane domain derived from CD28, 4-1BB/CD137, CD8 (e.g., CD8a), CD4, CD19, CD3 epsilon, CD45, CD5, CD9, CD16, CD22, CD33, CD37, CD64, CD80, CD86, CD134, CD137, CTLA4, PD-1, CD154, TCR alpha, TCR beta, or CD3 zeta and/or transmembrane regions containing functional variants thereof such as those retaining a substantial portion of the structural, e.g., transmembrane, properties thereof. Exemplary transmembrane domains are disclosed in WO2020227446; which is incorporated herein in its entirety.

Alternatively, the transmembrane domain in a CAR described herein may be synthetic, in which case it will comprise predominantly hydrophobic residues such as leucine and valine. Preferably a triplet of phenylalanine, tryptophan and valine will be found at each end of a synthetic transmembrane domain. A transmembrane domain of the invention is thermodynamically stable in a membrane. It may be a single alpha helix, a transmembrane beta barrel, a beta-helix of gramicidin A, or any other structure. Transmembrane helices are usually about 20 amino acids in length.

Optionally, a short oligo- or polypeptide linker, preferably between 2 and 10 amino acids in length may form the linkage between the transmembrane domain and the intracellular signaling domain(s) of the CAR. For example. A glycine-serine doublet may provide a suitable linker.

d. Costimulatory Domain

The cytoplasmic domains of a classic CAR construct trigger or elicit activation of at least one of the normal effector functions of the immune cell in which the CAR has been introduced. A classic CAR generally comprises one or more costimulatory domain and a primary activation/signaling domain which is a CD3ζ signaling domain in most CARs.

An CAR of the present invention does not comprise a primary intracellular activation/signaling domain. In one embodiment, the CAR of the present invention does not comprise a CD3ζ signaling domain.

In accordance, an CAR may comprise one or more costimulatory domains. As used herein, the term “costimulatory signaling domain,” or “costimulatory domain”, refers to an intracellular signaling domain derived from a co-stimulatory molecule. Costimulatory molecules are cell surface molecules other than antigen receptors or Fc receptors that provide a second signal required for efficient activation and function of T cells upon binding to antigen. Costimulatory signals are required to achieve robust chimeric antigen receptor (CAR) comprising cell expansion, function, persistence and antitumor activity.

In some embodiments, a costimulatory region according to the present invention is a signaling region of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD11a/CD18), CD3 gamma, CD3 delta, CD3 epsilon, CD247, CD276 (B7-H3), LIGHT (tumor necrosis factor superfamily member 14; TNFSF14), NKG2C, Ig alpha (CD79a), Fc gamma receptor, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAMI (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

In some cases, the design of an CAR may be optimized in light of the γδ TCR molecular structure and costimulation. The costimulatory domains promotes survival, proliferation and activation of γδ T cells.

e. Signal Domain

Optionally the CAR construct can comprise a signaling domain. The signaling domain can be natural or synthetic. In one aspect, the signal domain, e.g., an IgG1 signal peptide, is located at the beginning of the antigen binding domain of an CAR. Other signal peptides are known in the art.

f. Additional Polypeptides

In some embodiments, a CAR polypeptide further expresses one or more additional polypeptides. As non-limiting examples, the CAR polypeptide comprises a cytokine receptor. Cytokine regulation is critical for efficient skin γδ T cell localization, homeostatic turnover, and downstream function in the skin. For example, IL-2 and IL-7 are required for skin γδ T cell proliferation.

g. Linker

In some embodiments, the additional polypeptides can be linked to the CAR fusion protein (for example, to the costimulatory domain) using a linker.

In some embodiments, the linker is a peptide linker. A peptide linker refers to a plurality of amino acid residues between the various polypeptide domains added for appropriate spacing and conformation of the molecule. In some embodiments, a linker sequence separates one or more heavy or light chain variable domains of an antigen binding domain, hinge domains, transmembrane domains, co-stimulatory domains, and/or additional polypeptides. Exemplary linkers suitable for use in particular embodiments contemplated herein include GGGS (SEQ ID NO: 3), GGS or GS repeats.

In some embodiments, the linker can be a cleavable linker. In some embodiment, the linker is a non-cleavable linker.

In some embodiments, the linker is a self-cleaving peptide, e.g., 2A peptides. 2A peptides are a class of 18-22 aa-long peptides. Exemplary 2A peptides include P2A, T2A, E2A and F2A.

### Methods of Uses

In accordance with the present disclosure, compositions and γδ T cells comprising elevated expression of XCL1 and/or XCL2 can be used for immunotherapy, for example, increasing an immune response, therefore for the treatment of a cancer and other immune diseases.

In some embodiments, the elevated expression of XCL1 and/or XCL2 in the γδ T cells enhances the potential of the cells to significantly include downstream adaptive responses. In some embodiments, the elevated expression of XCL1 and/or XCL2 in the γδ T cells enhances the cells cytolytic reaction of the cell. In some embodiments, the elevated expression of XCL1 and/or XCL2 in the γδ T cells enhances the cytotoxicity, potency, proliferation and/or anti-tumor activity relative to a control immune cell.

In some embodiments, XCL1 and/or XCL2 expressing immune cells described herein may be used to target cDC1, recruiting cDC1 and increasing cDC1 accumulation in tumor tissue or local tissues. cDC1 accumulation in tumors can increase antigen cross-presentation, thereby increasing anti-tumor immune response. cDC1 cells further attract T cells, re-stimulate and expand tumor-specific CD8+ T cells, and support T cell effector function by secreting IL-12. Without limiting to any theory, the overexpression of XCL1 and/or XCL2 in γδ T cells may enhance adaptive immunity through increased recruitment of cDC1 to the tumor microenvironment.

In some embodiments, XCL1 and/or XCL2 expressing immune cells described herein attract NK cells and T cells to tumor tissue or other local tissues.

In some embodiments, γδ T cells comprising elevated expression of XCL1 and/or XCL2 are CAR modified γδ T cells. CAR modified γδ T cell based immunotherapy provides improved methods for treating cancer and other diseases. In accordance, the present invention provide improved methods of immunotherapy to fine-tune the safety and efficacy of a cytotoxic response against target cells, e.g., tumor cells, expressing target antigens while decreasing the risk of on-target antigen, off-target cell cytotoxicity. The inherent migration tropism of γδ T cells can increase T cells' or NK cells' targeting to cancer cells. In some embodiments, γδ T cells can act as a professional antigen presenting cells, combination therapies with other modalities of immunotherapy such as checkpoint inhibitors, oncolytic viruses, vaccines, or cytokines could synergistically amplify recruitment and function of tumor-infiltrating lymphoid and non-lymphoid cells.

### Therapeutic Methods

The present disclosure further provides a method of treating a patient in need thereof with cancer, infection or autoimmune disease, including administering to the patient an effective amount of the immune cell or the pharmaceutical composition according to the present disclosure.

In some embodiments, compositions and γδ T cells comprising elevated expression of XCL1 and/or XCL2 are used for the treatment of cancerous diseases, including tumorous diseases, including any malignancies that express the antigen. The administration of the composition(s) of the disclosure is useful for all stages (I, II, III, or IV) and types of cancer, including for minimal residual disease, early cancer, advanced cancer, and/or metastatic cancer and/or refractory cancer, for example. CAR and/or cytokine modified γδ T cells or NK-cells improve antitumor immune response. Examples of cancers that can be treated by the methods of the present invention include, but are not limited to, cancers of the immune system including lymphoma, leukemia, myeloma, and other leukocyte malignancies. In some embodiments, the “cancer” or “cancer tissue” comprises a solid tumor.

In some embodiments, cancers for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

As non-limiting examples, compositions and γδ T cells comprising elevated expression of XCL1 and/or XCL2 are used for treating B cell related diseases/conditions. B cell related conditions include but are not limited to immunoregulatory conditions and hematological malignancies. In some embodiments, the disease is B cell malignancy, which refers to a type of cancer that forms in B cells (a type of immune system cell) as discussed infra. The B-cell related disease may be selected from the group consisting of plasmacytoma, Hodgkin lymphoma, follicular lymphoma, small non-cutting nuclear cell lymphoma, endemic Burkitt lymphoma, sporadic Burkitt lymphoma, marginal zone lymphoma, extranodal mucosal lymphoma Tissue lymphoma, nodular monocytic B cell lymphoma, splenic lymphoma, mantle cell lymphoma, large cell lymphoma, diffuse mixed cell lymphoma, immunoblastic lymphoma, mediastinal primary B cell lymphoma, lung B cell angiocentric lymphoma, Small lymphocytic lymphoma, B cell of unknown malignancy, lymphoma-like granulomatosis, post-transplant lymphoproliferative disorder, immunoregulatory disease, rheumatoid arthritis, myasthenia gravis, idiopathic thrombocytopenia purpura, anti-Phospholipid syndrome, Chagas disease, Graves' disease, Wegener's granulomatosis, polyarteritis nodosa, Sjogren's syndrome, pemphigus vulgaris, scleroderma, Multiple sclerosis, antiphospholipid syndrome, ANCA-related vasculitis, Goodpasture disease, Kawasaki disease, autoimmune hemolytic anemia, rapidly progressive glomerulonephritis, heavy chain disease, and primary or immune cell amyloidosis.

In some embodiments, the cancer is a T-cell associated cancer. A T cell malignancy includes T-lymphoblastic lymphoma/leukemia, Cutaneous T-cell lymphomas (mycosis fungoides, Sezary syndrome, and others), Adult T-cell leukemia/lymphoma, Angioimmunoblastic T-cell lymphoma, extranodal natural killer/T-cell lymphoma, nasal type, Enteropathy-associated intestinal T-cell lymphoma (EATL), Anaplastic large cell lymphoma (ALCL), and Peripheral T-cell lymphoma, not otherwise specified (PTCL, NOS). An attenuated chimeric antigen receptor (CAR) comprising a T-cell antigen binding domain is disclosed for the treatment of various hematological malignancies including multiple myeloma. The T-cell antigen protein is expressed on a cancer cell. The antigen-binding portion of the CAR interacts with an epitope within the extracellular domain of the T-cell antigen fragment thereof.

In some embodiments, the present invention provides a method of inhibiting proliferation of or reducing a population of T-cell antigen-expressing cancer cells, the method comprising contacting a population of T-cell antigen-expressing cancer cells with γδ T-cells comprising elevated expression of XCL1 and/or XCL2 that express an attenuated CAR binding to the T cell antigen expressing cells. In certain aspects, the CAR and/or γδ T-cells comprising elevated expression of XCL1 and/or XCL2 reduce the number, amount, or percentage of cells and/or cancer cells in a subject or animal model of myeloid leukemia or another cancer associated with a T-cell antigen-expressing cell by at least 25%, at least 30%, at least 40%, at least 50%, at least 65%, at least 75%, at least 85%, at least 95%, or at least 99% relative to a negative control. In one embodiment, the subject is a human.

In some embodiments, a method of preventing, treating, or ameliorating at least one symptom of a cancer comprises administering the subject an effective amount of γδ T-cells comprising elevated expression of XCL1 and/or XCL2 comprising one or more attenuated CARs. The genetically modified cells are a more efficacious and safer cellular immunotherapy by virtue of transducing a chemically regulatable immunostimulatory signal.

In various embodiments, the treatment results in a decrease in the prevalence, frequency, level, and/or amount of one or more symptoms or biomarkers associated with the disease being treated, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms or biomarkers as compared to a prior measurement in the subject or to a reference value.

The quantity and frequency of administration of modified immune effector cells will be determined by such factors as the condition of the patient, and the type and severity of the patient's disease, although appropriate dosages and dose schedules may be determined by clinical trials.

One of ordinary skill in the art would recognize that multiple administrations of the compositions contemplated in particular embodiments may be required to effect the desired therapy. For example, a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more. Modified immune effector cells may be administered in the same or different compositions; in one or more compositions at the same time; or more than one composition at different times. Modified immune effector cells may be administered through the same route of administration or different routes.

The methods for administering the cell compositions contemplated herein include any method which is effective to result in reintroduction of ex vivo engineered γδ T cells. One method comprises modifying skin-derived γδ T cells ex vivo by introducing one or more vectors encoding a cytokine and/or CAR and returning the transduced cells into the subject.

### Combination Therapies

The disclosure further encompasses co-administration protocols with other compounds, e.g., bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, gene therapy, DNA therapy, viral therapy, RNA therapy, immunotherapy, bone marrow transplantation, monoclonal antibody therapy, or other types of immunotherapy.

According to the present disclosure, cytokines and/or CARs, vectors and cells expressing cytokines and/or CAR and compositions described herein can be used in combination with other therapies for cancer treatment. In some embodiments, the compositions and methods of the present embodiments involve an immune cell population (including T cell population or a γδ T-cell population or NK-cell population) in combination with at least one additional therapy.

In various embodiments, the cytokines and/or CARs, cells and compositions thereof may be included in a course of treatment that further includes administration of at least one additional agent to a subject.

In various embodiments, one or more additional agents may be administered at the same time, e.g., on the same day, or in the same week. In various embodiments, one or more additional agents may be administered in a single formulation with the present composition. In certain embodiments, an additional agent is administered in a manner temporally separated from administration of the present composition, e.g., one or more hours before or after, one or more days before or after, one or more weeks before or after, or one or more months before or after administration of the present composition. In various embodiments, the administration frequency of one or more additional agents may be the same as, similar to, or different from the administration frequency of the present composition.

In various embodiments, an additional agent administered in combination with the present composition may be a chemotherapy agent. For example, the composition can be administered to a subject at the same time, prior to, or after, chemotherapy.

In some embodiments, an immune cell therapy may be administered before, during, after, or in various combinations relative to an additional cancer therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

### Kits

Any of the cells and compositions described herein may be comprised in a kit. In a non-limiting example, cells, reagents to produce cells, vectors, and reagents to produce vectors and/or components thereof may be comprised in a kit. In certain embodiments, γδ T-cells comprising elevated expression of XCL1 and/or XCL2 are comprised in a kit, and they may or may not yet express a CAR. In some embodiments, the γδ T-cells comprising elevated expression of XCL1 and/or XCL2 additionally express IL-2. In some embodiments, such a kit comprises one or more reagents for manipulation of cells. Such reagents include small molecules, proteins, nucleic acids, antibodies, buffers, primers, nucleotides, salts, and/or a combination thereof, for example. Nucleotides that encode one or more CARs, suicide gene products, and/or cytokines may be included in the kit. Proteins, such as cytokines or antibodies, including monoclonal antibodies, may be included in the kit. Nucleotides that encode components of engineered CARs may be included in the kit, including reagents to generate same.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified in particular embodiments to yield essentially similar results.

### EXAMPLES

While certain compounds, compositions and methods of the present disclosure have been described with specificity in accordance with certain embodiments, the following examples serve only to illustrate the disclosure and are not intended to limit the same.

### Example 1: Bulk RNA Sequencing to Assess Vδ1+ T Cells Transcriptome

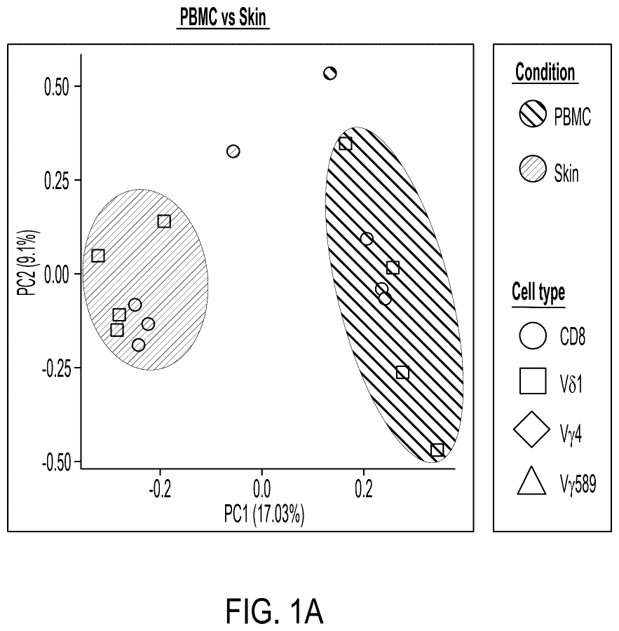

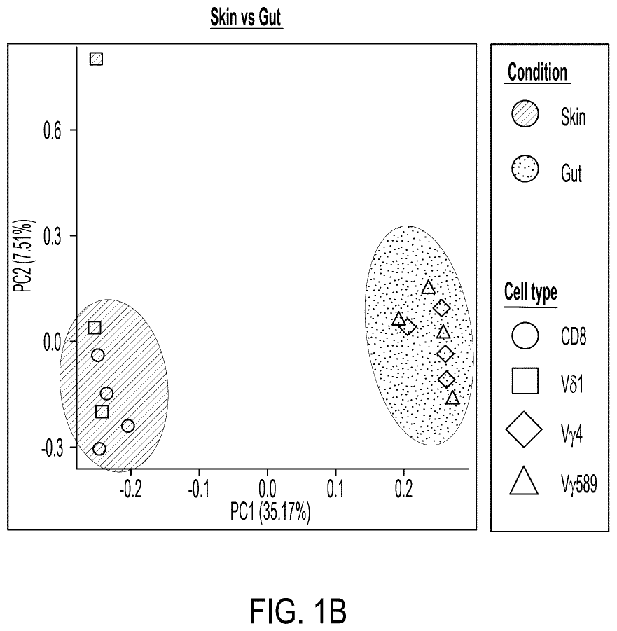

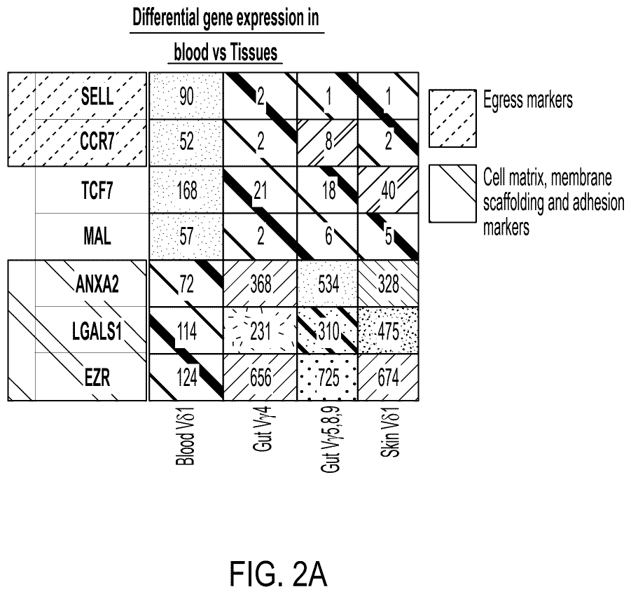

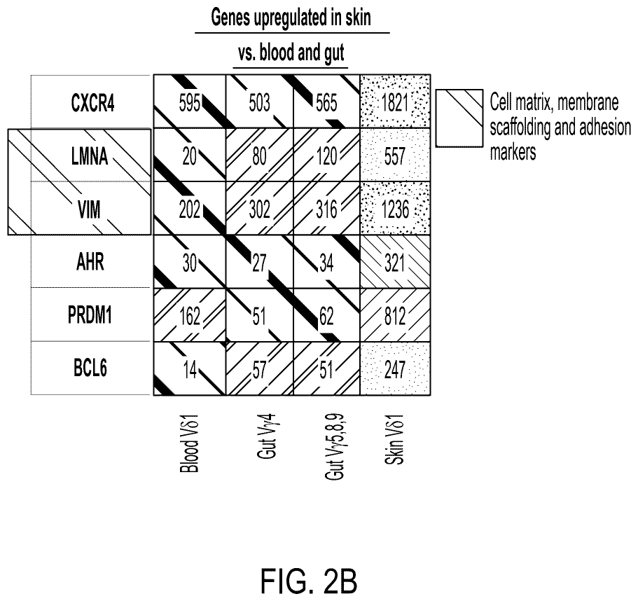

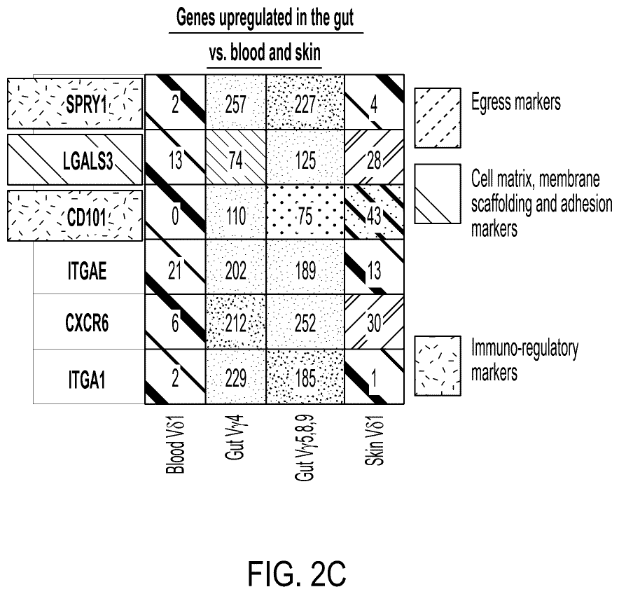

The transcriptome of human, skin derived Vδ1+ T cells was compared and contrasted to their counterparts from the blood and colon, enabling insights into the unique properties of these cells and the tissue-specific adaptations they display. RNA sequencing was used to assess the transcriptome of human skin derived Vδ1+ cells and their counterparts from the blood and the colon. Key components of the data were validated at the protein level by flow cytometry or ELISA. Principal Component Analysis (PCA) of samples derived from blood and skin (FIG. 1A) and skin and gut (FIG. 1B) were submitted for bulk RNA sequencing.

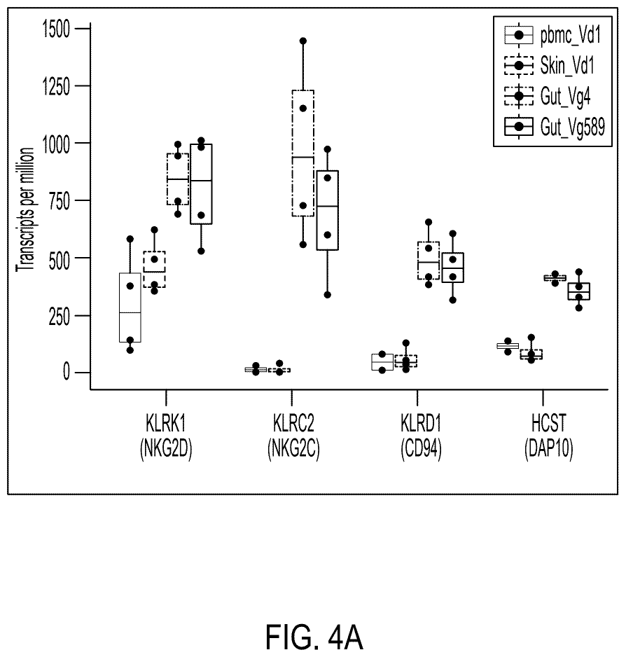

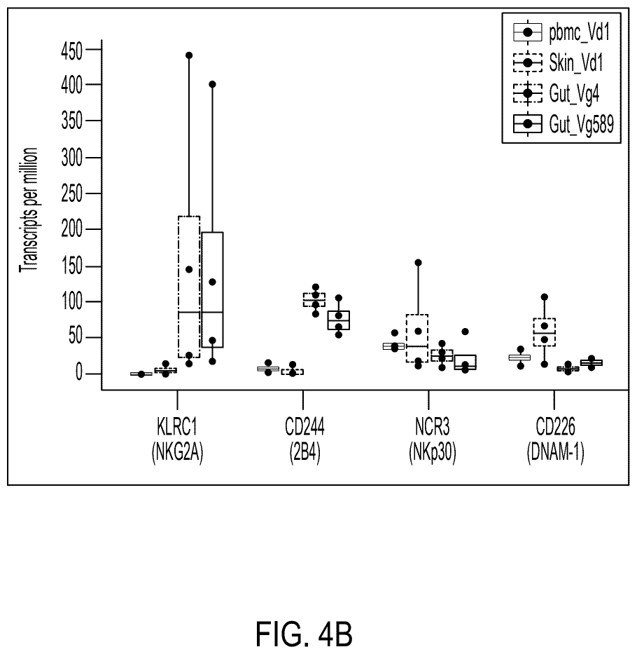

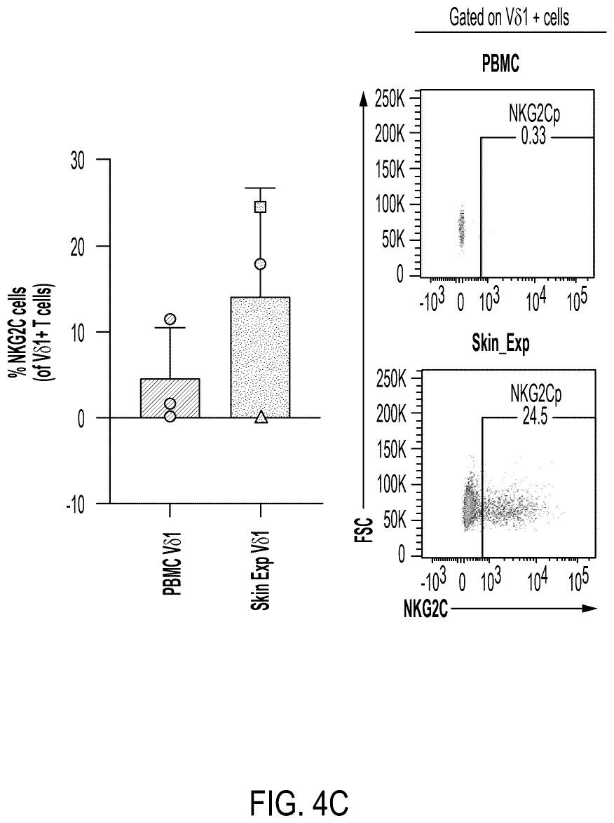

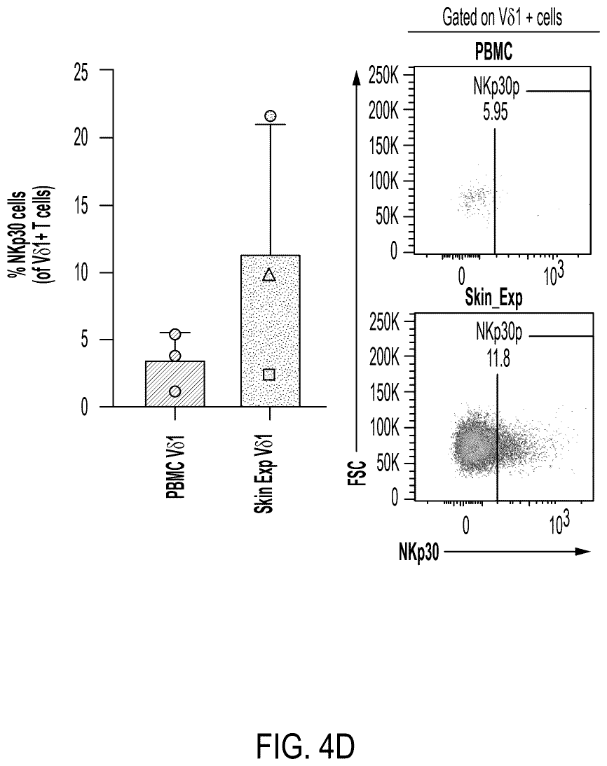

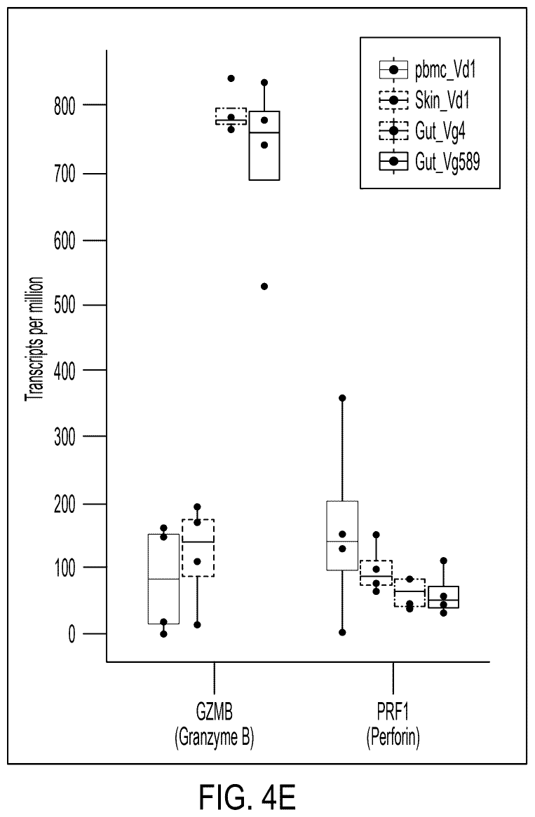

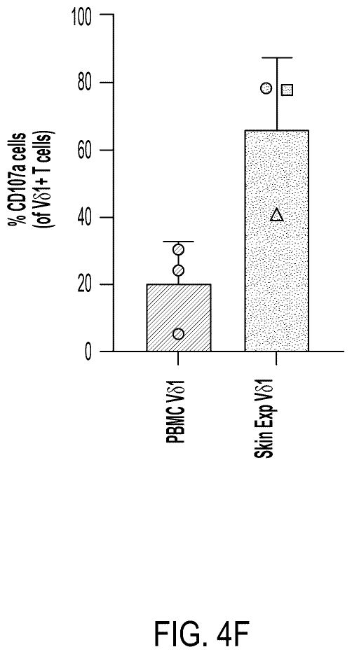

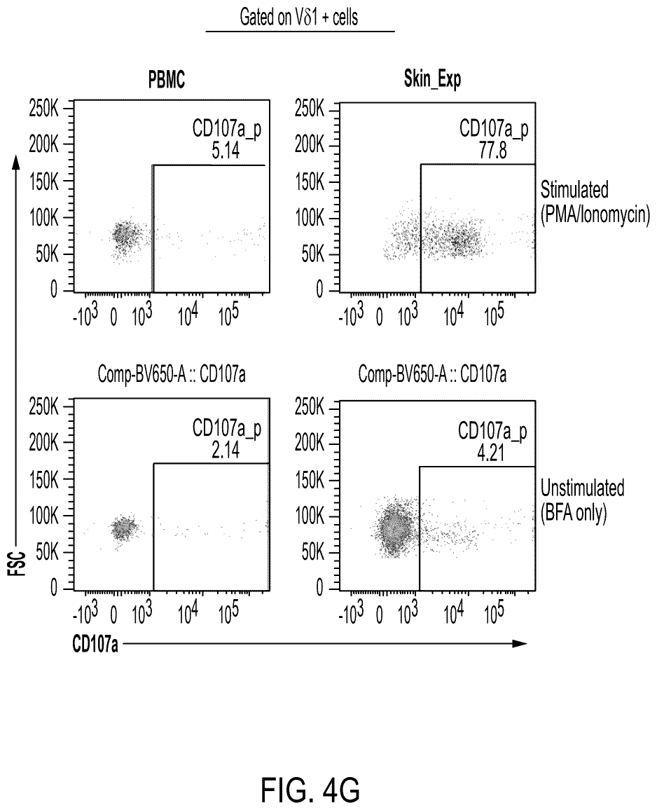

PCA demonstrated that anatomical location impacts T cell transcriptomic signatures. The data revealed distinctive transcriptional signatures characterizing human skin derived Vd1+ T cell populations, demonstrating a clear aptitude for immunosurveillance. These populations exhibit marked attributes such as tissue-homing, skewed effector function, and immune orchestration. This investigation into skin, blood, and gut tissues unveiled unique properties of tissue-specific gd T cells. Both Vd1 cells from blood and skin, as well as Vg4+Vg5,8,9 cells from the gut, demonstrated a cytotoxic profile. Notably, the gut derived cells exhibited the highest transcript levels of genes associated with cytotoxicity (FIG. 4).

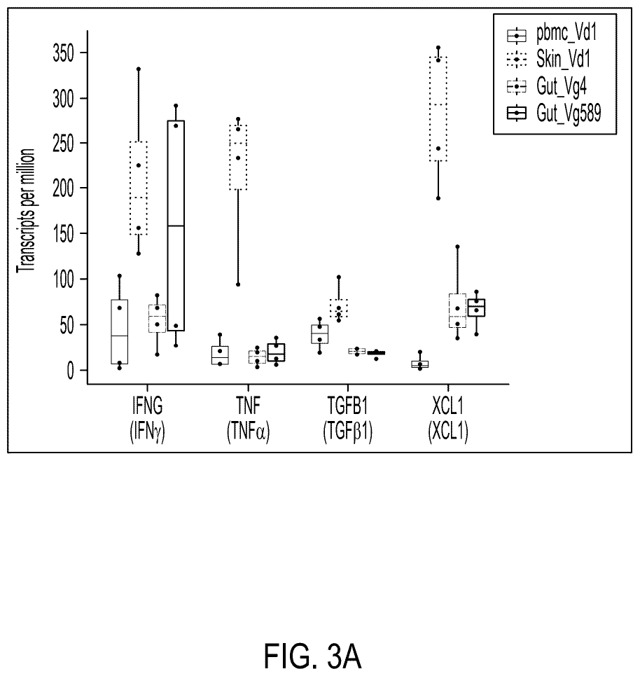

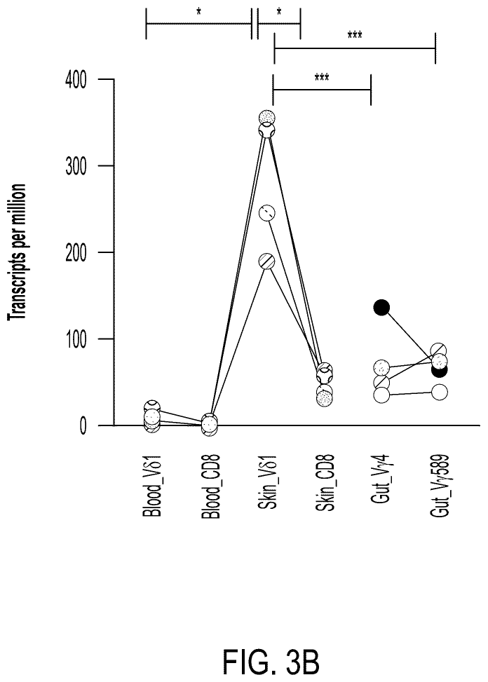

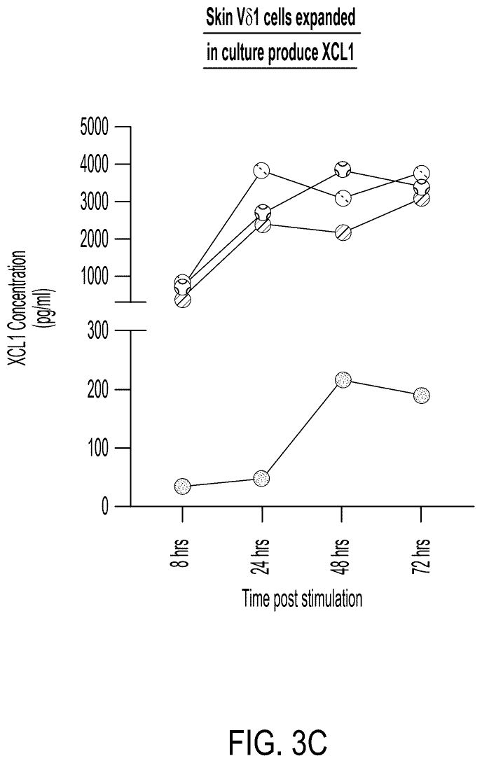

Fundamental differences between skin Vd1+ cells and their counterparts in blood and the gut were observed. Skin Vd1+ cells emerged as an innate-like subset, characterized by a Tc1-type cytokine profile, as evidenced by elevated transcript levels of IFNg and TNFα (FIG. 3A). Moreover, these skin derived cells expressed particularly high levels of XCL1 (FIG. 3A-B3), endowing them with the potential to significantly influence downstream adaptive responses.

### EQUIVALENTS AND SCOPE

Those skilled in the art will recognize or be able to ascertain using no more than routine experimentation, many equivalents to the specific embodiments of the invention described herein. The scope of the present invention is not intended to be limited to the above description, but rather is as set forth in the following claims:

1. An engineered immune cell expressing XCL1 (X-C motif chemokine ligand 1) or a functional variant thereof, wherein the immune cell is a gamma delta (γδ) T cell.

2. The engineered immune cell of claim 1, wherein the γδ T cell is a tissue-derived γδ T cell or a blood-derived γδ T cell.

3. The engineered immune cell of claim 2, wherein the γδ T cell is a skin-derived γδ T cell.

4. The engineered immune cell of claim 2, wherein the γδ T cell is a blood-derived γδ T cell.

5. The engineered immune cell of any one of claims 1-4, wherein the γδ T cell is Vdelta1(Vδ1+) T cell.

6. The engineered immune cell of claim 5, wherein the immune cell is a human skin-derived Vδ1+ T cell.

7. The engineered immune cell of any one of the preceding claims, wherein the engineered immune cell expresses an increased level of XCL1 or the functional variant thereof, as compared to the expression level of XCL1 in a control immune cell.

8. The engineered immune cell of any one of the preceding claims, wherein NKG2C and NKp30 expression is elevated.

9. The engineered immune cell of any one of the preceding claims, wherein IFNγ, TNFα and TGFβ1 expression is elevated.

10. The engineered immune cell of any one of the preceding claims, wherein the immune cell has an increased cytolytic reaction.

11. The engineered immune cell of any one of the preceding claims, wherein the engineered immune cell further expresses at least one chimeric antigen receptor (CAR).

12. The engineered immune cell of any one of the preceding claims, wherein the immune cell has greater cytotoxicity, potency, proliferation and/or anti-tumor activity relative to a control immune cell.

13. The engineered immune cell of any one of the preceding claims, wherein the XCL1, or functional variant thereof is expressed under an inducible promoter.

14. The engineered immune cell of any one of claims 1-13, wherein the XCL1 or the functional variant thereof is expressed under a constitutive promoter.

15. The engineered immune cell of any one of the preceding claims, wherein the γδ T cell is isolated from a tissue or derived from a pluripotent stem cell.

16. The engineered immune cell of claim 15, wherein the γδ T cell is isolated from skin.

17. The engineered immune cell of any one of the preceding claims, wherein the immune cell is allogeneic.

18. The engineered immune cell of any one of the preceding claims, wherein the immune cell is further expanded.

19. An immune cell comprising elevated expression of XCL1 as compared to a control immune cell, wherein the immune cell is a γδ T cell.

20. The immune cell of claim 19, wherein XCL1 is overexpressed.

21. The Immune cell of claim 19, wherein the immune cell is modified to express a XCL1 or a functional variant thereof.

22. The immune cell of any one of claims 19-20, wherein the γδ T cell is Vdelta1(Vδ1+) T cell.

23. The immune cell of any one of claims 19-22, wherein the γδ T cell is a blood-derived or tissue-derived Vδ1+ T cell.

24. The immune cell of any one of claims 19-23, wherein the γδ T cell is expanded.

25. A composition comprising the engineered immune cell of any one of claims 1-18 or the immune cell of any one of claims 19-24.

26. A method of modulating immune function comprising administering to a subject in need thereof the engineered immune cell of claims 1-18, the immune cell of any one of claims 19-24, or the composition of claim 25.

27. The method of claim 26, wherein administration of the immune cell leads to increased proliferation of immune cells, cytotoxicity, potency and/or tumor killing.

28. The method of claim 27, wherein the immune cell activates XCR1+ dendritic cells (cDC1), T cells and/or NK cells.

29. The method of any one of claims 26-28, wherein the immune cell increases immunosurveillance in the subject.

30. A method of treating cancer comprising administering to a patient in need thereof a therapeutically effective amount of the engineered immune cell of any one of claims 1-18, the immune cell of any one of claims 19-24, or the composition of claim 25.

31. The method of claim 30, wherein the administration increases recruitment of cDC1 cells to tumor tissues, thereby increasing anti-cancer immune response.

32. The method of claim 30 or 31, wherein the patient is further treated with another anti-cancer therapy.


SEQUENCE LISTING (3 sequences)

SEQ ID NO: 1 - protein, 114 aa, synthetic construct
MRLLILALLGICSLTAYIVEGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 2 - protein, 114 aa, synthetic construct
MRLLILALLGICSLTAYIVEGVGSEVSHRRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 3 - protein, 4 aa, synthetic construct
GGGS